(19) United States
(12) Patent Application Publication
(10) Pub. No.: US 2025/0241591 A1
(43) Pub. Date: Jul. 31, 2025

(54) SENSOR DEVICE AND SENSING METHOD

(71) Applicant: Japan Display Inc., Tokyo (JP)

(72) Inventors: Naoki TAKADA, Tokyo (JP); Adithya NARESH, Hayward, CA (US)

(73) Assignee: Japan Display Inc., Tokyo (JP)

(21) Appl. No.: 18/422,031

(22) Filed: Jan. 25, 2024

(51) Int. Cl. A61B 5/00

(52) U.S. Cl. CPC A61B 5/6844; A61B 5/681; A61B 2560/0209; A61B 2560/0214

(57) ABSTRACT

According to one embodiment, a sensor device includes a sensor, a control unit configured to cause the sensor to perform sensing and receive a signal from the sensor, and a processor configured to determine presence or absence of a sensing and obtain biometric information of the sensing target. The control unit is configured to cause the sensor to perform first sensing and cause the sensor to perform second sensing when the processor determines the presence of the sensing target during the first sensing. Power consumption during the first sensing is less than power consumption during the second sensing.

### FIELD

Embodiments described herein relate generally to a sensor device and a sensing method.

### BACKGROUND

One example of a sensor device is a sensor device that irradiates light onto a sensing target. The sensor device detects reflected light or transmitted light from the sensing target. The sensor device operates on a rechargeable battery. Power-saving operation is desired for the sensor device.

### BRIEF DESCRIPTION OF THE DRAWINGS

FIG. 1 is illustrates an example of a sensor system according to a first embodiment.

FIG. 2 illustrates an example of an arrangement a sensor panel and a light source according to the first embodiment in the case of a reflective sensor.

FIG. 3 illustrates an example of an arrangement a sensor panel and a light source according to the first embodiment in the case of a transmissive sensor.

FIG. 4 illustrates an example of a sensor panel and a sensor control unit according to the first embodiment.

FIG. 5 illustrates details of an example of a sensor panel and a sensor control unit according to the first embodiment.

FIG. 6 is a signal waveform diagram to illustrate an example of an operation of a sensor device according to the first embodiment.

FIG. 7 illustrates an example of a light 1 emission timing of a light source according to the first embodiment.

FIG. 8 shows an example of mounting a sensor device including a sensor panel and a light source to a sensing target according to the first embodiment.

FIG. 9 shows an example of acquiring a sensor signal according to the first embodiment.

FIG. 10 shows an example of change in a waveform of an output voltage of a buffer amplifier.

FIG. 11 shows another example of change in a waveform of an output voltage of a buffer amplifier.

FIG. 12 shows a still another example of change in a waveform of an output voltage of a buffer amplifier.

FIG. 13 is a diagram illustrating an example of an overview of an operation of a sensor device according to the first embodiment.

FIG. 14A, FIG. 14B, and FIG. 14C illustrate an example of details of the operation of the sensor device according to the first embodiment.

FIG. 15A, FIG. 15B, and FIG. 15C illustrate an example of details of the operation of the sensor device according to the first embodiment.

FIG. 16 shows an example of mounting a sensor device including a sensor panel and a light source to a sensing target according to a second embodiment.

FIG. 17 shows an example of acquiring a sensor signal according to the second embodiment.

### DETAILED DESCRIPTION

Various embodiments will be described hereinafter with reference to the accompanying drawings.

The disclosure is merely an example and is not limited by contents described in the embodiments described below. Modification which is easily conceivable by a person of ordinary skill in the art comes within the scope of the disclosure as a matter of course. In order to make the description clearer, the sizes, shapes, and the like of the respective parts may be changed and illustrated schematically in the drawings as compared with those in an accurate representation. Constituent elements corresponding to each other in a plurality of drawings are denoted by like reference numerals and their detailed descriptions may be omitted unless necessary. “Connection” is not limited to direct connection and may include indirect connection through another element.

In general, according to one embodiment, a sensor device comprising:
a sensor configured to detect reflected light from a sensing target or transmitted light of the sensing target;
a control unit configured to cause the sensor to perform sensing and receive a detection signal from the sensor; and
a processor configured to determine presence or absence of the sensing target based on a detection signal of the sensor and obtain biometric information of the sensing target based on fluctuations in the detection signal over a certain period of time, wherein the control unit is configured to cause the sensor to perform first sensing;
the control unit is configured to cause the sensor to perform second sensing when the processor determines
the presence of the sensing target during the first sensing; and
power consumption during the first sensing is less than power consumption during the second sensing.

### First Embodiment

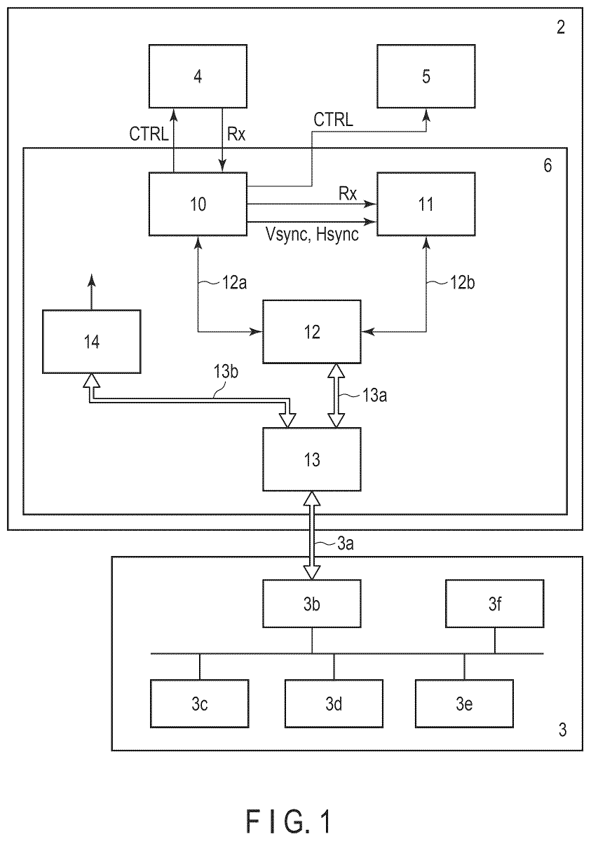

FIG. 1 illustrates an example of a sensor system according to a first embodiment. The sensor system comprises a sensor device 2 and a PC (personal computer) 3. The sensor device 2 is connected to the PC 3. An example of a connection line between the sensor device 2 and the PC 3 is a USB cable 3a. The sensor device 2 and the PC 3 may be connected to each other wirelessly.

The sensor device 2 comprises a sensor panel 4, a light source 5, and a sensing circuit 6. The light source 5 irradiates light onto a sensing target. An example of a sensing target is a hand (specifically an artery). An example of light is infrared light. The type of sensor may be reflective or transmissive. In a case where the sensor type is reflective, reflected light from the sensing target is incident on the sensor panel 4. In a case where the sensor type is transmissive, transmitted light from the sensing target is incident on the sensor panel 4. The sensor panel 4 may comprise a photodiode as an optical sensor element. The sensor panel 4 may comprise a single photodiode or a plurality of photodiodes arranged in a one-dimensional or two-dimensional array. The photodiodes are formed on a substrate. A collimator lens is placed on the photodiode. The sensor device 2 obtains the state of the sensing target and biometric information of a sensing subject from the reflected or transmitted light of the artery.

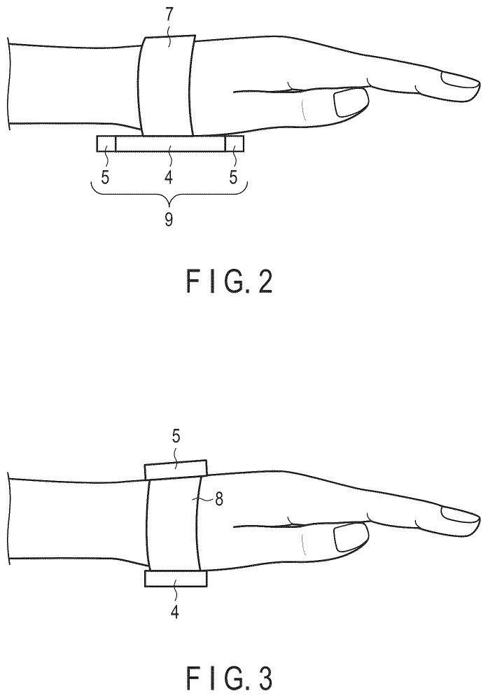

FIG. 2 illustrates an example of an arrangement of the sensor panel 4 and the light source 5 according to the first embodiment in the case of a reflective sensor. The light source 5 is arranged on the same substrate as the sensor panel 4. The sensor panel 4 and the light source 5 are attached to a hand, wrist, etc. by a belt 7. The sensor panel 4 and the light source 5 are placed on the same side of the hand, wrist, etc. as the sensing target. The reflective sensor panel 4 and light source 5 may be configured like a wristwatch in the case of being worn on the back of the hand.

FIG. 3 illustrates another example of an arrangement of the sensor panel 4 and the light source 5 according to the first embodiment in the case of a transmissive sensor. The sensor panel 4 and the light source 5 are attached to a wrist, etc. by a belt 8 so as to sandwich the wrist. The sensor panel 4 and the light source 5 are arranged on opposite sides to the sensing target. The sensor panel 4 and the light source 5 may be worn so as to sandwich a finger.

Returning to the description of FIG. 1, the sensing circuit 6 comprises a sensor control unit 10, a signal processor 11, a micro controller unit (MCU) 12, a USB IF circuit 13, and a power control unit 14.

The MCU 12 is connected to the sensor control unit 10 via a serial communication line 12a. The MCU 12 is connected to the signal processor 11 via a serial communication line 12b. Examples of a serial communication IF are SPI and I2C. The USB IF circuit 13 is connected to the MCU 12 via a parallel communication line 13a. The USB IF circuit 13 is connected to the power control unit 14 via a parallel communication line 13b.

The power supply control unit 14 comprises a main switch, a rechargeable battery, and a power management IC (PMIC). The PMIC charges the rechargeable battery using a power supply voltage supplied by the PC 3 via the USB cable 3a, the USB IF circuit 13, and the parallel communication line 13b. The PMIC controls the charging of the rechargeable battery and generates multiple power supply voltages for the units in the sensing circuit 6 from the voltage of the rechargeable battery.

The sensor control unit 10 supplies a power supply voltage and a control signal (CTRL) to the light source 5. An example of the control signal is a pulse width modulation (PWM) signal. The light source 5 emits light in response to the control signal. The sensor control unit 10 supplies a power supply voltage and a control signals (CTRL) to the sensor panel 4. The sensor panel 4 senses the sensing target in response to the control signal and transmits an analog signal Rx (hereinafter referred to as a sensor signal) indicating the sensing result of each photodiode to the sensor control unit 10.

The sensor control unit 10 generates the PWM signal based on a change of the sensor signal Rx. If the sensor signal Rx represents that a sense result changes to a brighter side, the sensor control unit 10 generates the PWM signal for decreasing an intensity of the light source 5. If the sensor signal Rx represents that a sense result changes to a darker side, the sensor control unit 10 generates the PWM signal for increasing an intensity of the light source 5. The sensor control unit 10 may generate the PWM signal based on a generation control signal from a host (not shown). The sensor control unit 10 transmits the sensor signal Rx as it is to the signal processor 11. The sensor control unit 10 transmits a vertical synchronization signal Vsync and a horizontal synchronization signal Hsync to the signal processor 11. The sensor control unit 10 converts the sensor signal Rx into digital sensor data and transmits the sensor data to the MCU 12 via the serial communication line 12a.

The signal processor 11 acquires the sensor signal Rx of the photodiode at a desired position according to the synchronization signals Vsync and Hsync. By processing the sensor signal Rx, the signal processor 11 obtains position data indicating the position of the sensor signal Rx (the position of the photodiode from which the sensor signal Rx was acquired), status data indicating the state of the sensing target, and biometric data indicating biometric information of the sensing subject. Examples of the state of the sensing target are the presence or absence of the sensing target, presence or absence of a pulse wave, and the presence or absence of breathing. Examples of the biometric information are pulse rates and breathing rates. The signal processor 11 transmits the position data, status data, and biometric data to the MCU 12 via the serial communication line 12b.

The MCU 12 transmits a mode control signal to the sensor control unit 10 via the serial communication line 12a according to the position data, status data, and biometric data transmitted from the signal processor 11. The sensor control unit 10 drives the sensor panel 4 according to the mode control signal. The MCU 12 transmits the sensor data transmitted from the sensor control unit 10 to the USB IF circuit 13 via the parallel communication line 13a.

The USB IF circuit 13 transmits the sensor data transmitted from the MCU 12 to the PC 3 via the USB cable 3a.

The PC 3 comprises a USB IF circuit 3b, a CPU 3c, a ROM 3d, a RAM 3e, and a communication circuit 3f. The USB IF circuit 3b, the CPU 3c, the ROM 3d, the RAM 3e, and the communication circuit 3f are connected to each other via a bus line. The USB IF circuit 3b is connected to the USB cable 3a. The CPU 3c executes a program stored in the ROM 3d. The RAM 3e stores data during program execution. The communication circuit 3f is connected to a network (not shown) and can be connected to a host connected to the network.

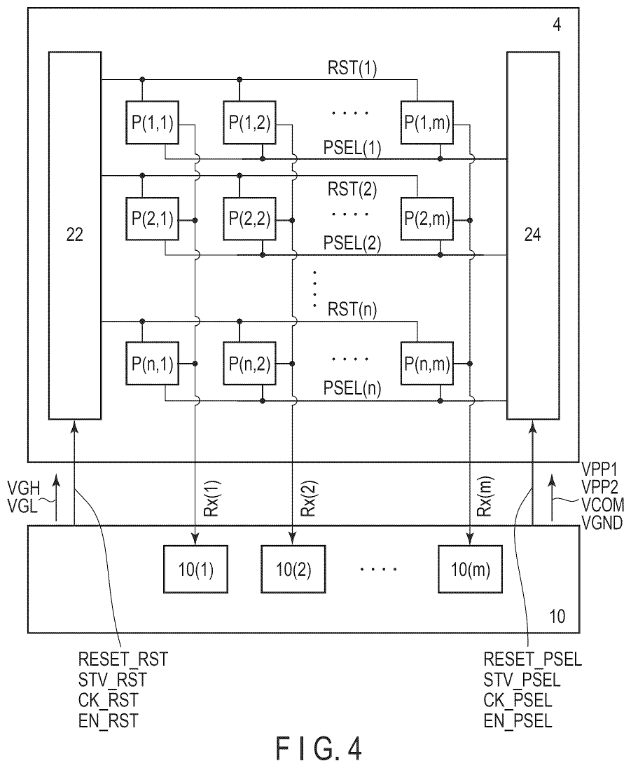

FIG. 4 illustrates an example of the sensor panel 4 and the sensor control unit 10 according to the first embodiment. The sensor panel 4 includes a plurality of sensor units P(1,1) to P(n,m), a reset (RST) gate driver 22, and a sensor unit selection (PSEL) gate driver 24. The plurality of sensor units P(1,1) to P(n,m) are arranged in a two-dimensional array of n rows by m columns. N and m are any positive integer.

The sensor units P(1,1) to P(1,m) are arranged in the first row of the two-dimensional array. The sensor units P(2,1) to P(2,m) are arranged in the second row of the two-dimensional array. Thereafter, the sensor units P in a row are arranged in the same manner. The sensor units P(n, 1) to P(n,m) are arranged in the nth row of the two-dimensional array.

The sensor units P(1, 1) to P(n, 1) are arranged in the first column of the two-dimensional array. The sensor units P(1,2) to P(n, 2) are arranged in the second column of the two-dimensional array. Thereafter, the sensor units P in a column are arranged in the same manner. The sensor units P(1,m) to P(n,m) are arranged in the m-th column of the two-dimensional array.

The RST gate driver 22 has n output terminals that output n reset pulses RST, respectively. Each of the n reset pulses RST is supplied to the sensor units P in each row. A reset pulse RST(1) output from the RST gate driver 22 is supplied to the sensor units P(1,1) to P(1,m) in the first row. A reset pulse RST(2) output from the RST gate driver 22 is supplied to the sensor units P(2,1) to P(2,m) in the second row. Thereafter, a reset pulse RST is supplied to the sensor units P in a row. A reset pulse RST(n) output from the RST gate driver 22 is supplied to the sensor units P(n, 1) to P(n,m) in the nth row.

The PSEL gate driver 24 has n output terminals that output n selection signals PSEL, respectively. Each of the n selection signals PSEL of the PSEL gate driver 24 is supplied to the sensor units P in each row. A selection signal PSEL(1) output from the PSEL gate driver 24 is supplied to the sensor units P(1,1) to P(1,m) in the first row. A selection signal PSEL(2) output from the PSEL gate driver 24 is supplied to the sensor units P(2,1) to P(2,m) in the second row. Thereafter, a selection signal PSEL is supplied to the sensor units P in a row. A selection signal PSEL(n) output from the PSEL gate driver 24 is supplied to the sensor units P(n, 1) to P(n,m) in the nth row.

The sensor control unit 10 includes m control units 10(1) to 10(m). Each of the control units 10(1) to 10(m) processes signals from the sensor units P in each column. A sensor signal Rx (1) of each of the sensor units P(1,1) to P(n, 1) in the first column of the two-dimensional array is supplied to the control unit 10(1). The sensor units P selected sequentially by the selection signal PSEL in the sensor units P(1, 1) to P(n, 1) output the sensor signals Rx sequentially. A sensor signal Rx (2) of each of the sensor units P(1,2) to P(n, 2) in the second column of the two-dimensional array is supplied to the control unit 10(2). Thereafter, a sensor signal Rx of each of the sensor units P in a column is supplied to the control unit 10. A sensor signal Rx (m) of the sensor units P(1,m) to P(n,m) in the m-th column of the two-dimensional array is supplied to the control unit 10(m).

The sensor signals Rx of the plurality of sensor units P(1,1) to P(n,m) form a frame.

In addition to the control units 10(1) to 10(m), the sensor control unit 10 includes a signal supply unit that supplies a power supply voltage and a control signal to the sensor units P. The sensor control unit 10 supplies two power supply voltages VGH and VGL to the RST gate driver 22 and the PSEL gate driver 24. The sensor control unit 10 supplies four power supply voltages VPP1, VPP2, VCOM, and VGND to the sensor units P. The sensor control unit 10 supplies four control signals RESET_RST, STV_RST, CK_RST, and EN_RST to the RST gate driver 22. The sensor control unit 10 supplies four control signals RESET_PSEL, STV_PSEL, CK_PSEL, and EN_PSEL to the PSEL gate driver 24.

Each of the signals RESET_PSEL and RESET_RST is a frame reset signal for resetting the sensor device once per frame. Each of the signals STV_PSEL and STV_RST is a frame start signal and is output once per frame. One frame is a period during which the sensor signals of all sensor units P are output. Each of the signals CK_PSEL and CK_RST is a clock signal for selecting a row of the two-dimensional array. Each of the signals EN_PSEL and EN_RST is an enable signals for causing the sensor unit P to generate a photocurrent in response to incident light, that is, for exposing the sensor unit P to light. The sensor unit P outputs the sensor signal Rx during exposure.

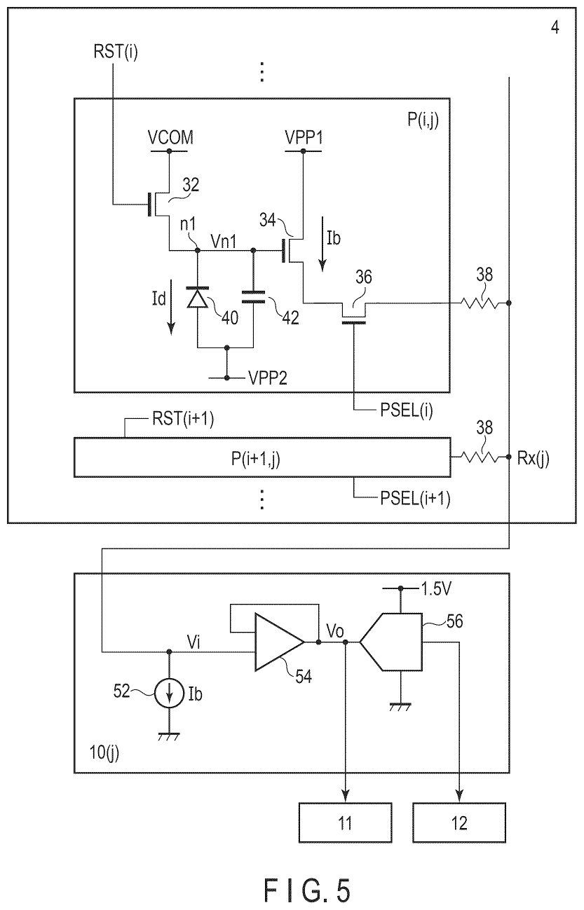

FIG. 5 illustrates details of an example of the sensor panel 4 and the sensor control unit 10 according to the first embodiment. All sensor units P have the same configuration. All control units 10 have the same configuration. FIG. 5 shows, as an example, a sensor unit P(i,j) and a control unit 10(j). A variable “I” is any positive integer from 1 to n. A variable “j” is any positive integer from 1 to m.

The sensor unit P(i,j) is a three-transistor and one-diode voltage detection type optical sensor. The transistors can be bipolar transistors or metal oxide semiconductor type field effect transistors (MOSFETS). FIG. 5 shows an example where a MOSFET is used as a transistor. The sensor unit P(I,j) includes MOSFETs 32, 34, and 36 and a photodiode 40.

The power supply voltage VCOM is applied to the drain of the MOSFET 32 of all sensor units P. A reset pulse RST(i) is supplied to the gate of the MOSFET 32 of sensor units P(i,1) to P(i,m) in an i-th row. A reset pulse RST(i+1) is supplied to the gate of the MOSFET 32 of sensor units P(i+1,1) to P(i+1,m) in an (i+1)th row. The source of the MOSFET 32 is connected to the gate of the MOSFET 34.

The source of the MOSFET 32 (the gate of MOSFET 34) is connected to the cathode of the photodiode 40. The cathode of the photodiode 40 is referred to as a node n1. A power supply voltage VPP2 is applied to the anode of the photodiode 40 of all sensor units P. A capacitor 42 is connected in parallel to the photodiode 40. The capacitor 42 is charged by a photocurrent generated by the photodiode 40.

A power supply voltage VPP1 is applied to the drain of the MOSFET 32 of all sensor units P. The source of the MOSFET 34 is connected to the drain of the MOSFET 36. A selection signal PSEL(i) is supplied to the gate of the MOSFET 36 of the sensor units P(i,1) to P(i,m) in the i-th row. A selection signal PSEL(i+1) is supplied to the gate of the MOSFET 36 of sensor units P(i+1,1) to P(i+1,m) in the (i+1)th row. The source of the MOSFET 36 of sensor units P(1, j) to P(n,j) in the jth column is an output terminal of the sensor unit P that outputs a sensor signal Rx(j).

The sensor unit P(i,j) in the jth column of the i-th row selected by the selection signal PSEL(i) outputs the sensor signal Rx(j). The sensor signal Rx(j) is supplied to an input terminal of the control unit 10(j) via a resistor 38. The resistor 38 is a resistor of a wiring line that transmits the sensor signal Rx(j) from the sensor unit P(i,j) to the control unit 10(j).

The MOSFET 34 is a source follower circuit. A drain-to-source current Ib of the MOSFET 34 is represented by Equation 1.

$$Ib = ( 1 / 2 ) \times {α ( Vgs - Vth )}^{2} \qquad Equation 1$$

$$α = μ \times Cox \times ( W / L ) \qquad Equation 2$$

Vgs is a gate-source voltage of the MOSFET 34. Vth is a threshold voltage of the MOSFET 34. “μ” is the electron mobility of the MOSFET 34. Cox is the gate oxide film capacitance per unit area of the MOSFET 34. W/L is a MOSFET 34-specific coefficient.

The control unit 10 includes a current source 52, a buffer amplifier 54, and an A/D converter (ADC) 56. The current source 52 is connected to the input terminal of the control unit 10. The current source 52 is the current source for the drain-to-source current Ib of the MOSFET 34.

An input voltage Vi of the control unit 10 is input to the buffer amplifier 54. An output voltage Vo of the buffer amplifier 54 is supplied to the signal processor 11. The output voltage Vo of the buffer amplifier 54 is supplied to the MCU 12 via the A/D converter 56.

The MOSFET 36 of the sensor units P(i,1) to P(i,m) in the i-th row is turned on or off according to the level of the selection signal PSEL(i). While the MOSFET 36 is on, the buffer amplifier 54 outputs a voltage Vo (=Vi) expressed by Equation 3.

$$Vo ( = Vi ) = ( Vn 1 - Δ Vn 1 ) - Vth - {( ( 2 / α ) Ib )}^{1 / 2} - IbR \qquad Equation 3$$

Vn1 is a voltage at the node n1. ΔVn1 is a voltage at the node n1 that decreases due to the photocurrent (reverse bias current) Id flowing through the photodiode 40. R is a resistance value of the resistor 38. If a capacitance of the capacitor 42 is represented by Cpix, an amount of electric charges in the capacitor 42 is expressed by ΔQ (coulomb) and ΣId is a total current flowing through the diode 40 during an exposure period, ΔVn1 is expressed by equation 4.

$$Δ Vn 1 = Δ Q / Cpix = \sum Id / Cpix \qquad Equation 4$$

The MOSFET 32 of the sensor units P(i,1) to P(i,m) in the i-th row is turned on or off according to the level of the reset pulse RST(i). While the MOSFET 32 is on, the buffer amplifier 54 outputs the voltage Vo (=Vi) expressed by Equation 5.

$$Vo ( = Vi ) = Vn 1 - Vth - {( ( 2 / α ) Ib )}^{1 / 2} - IbR \qquad Equation 5$$

The buffer amplifier 54 outputs ΔVo, which is a difference between Vo (=Vi) while the MOSFET 36 is on and Vi (=Vo) while the MOSFET 32 is on. ΔVo is a voltage of the sensor signal output from the sensor unit P(i,j). ΔVo is expressed by Equation 6.

$$Δ Vo = Δ Vni \qquad Equation 6$$

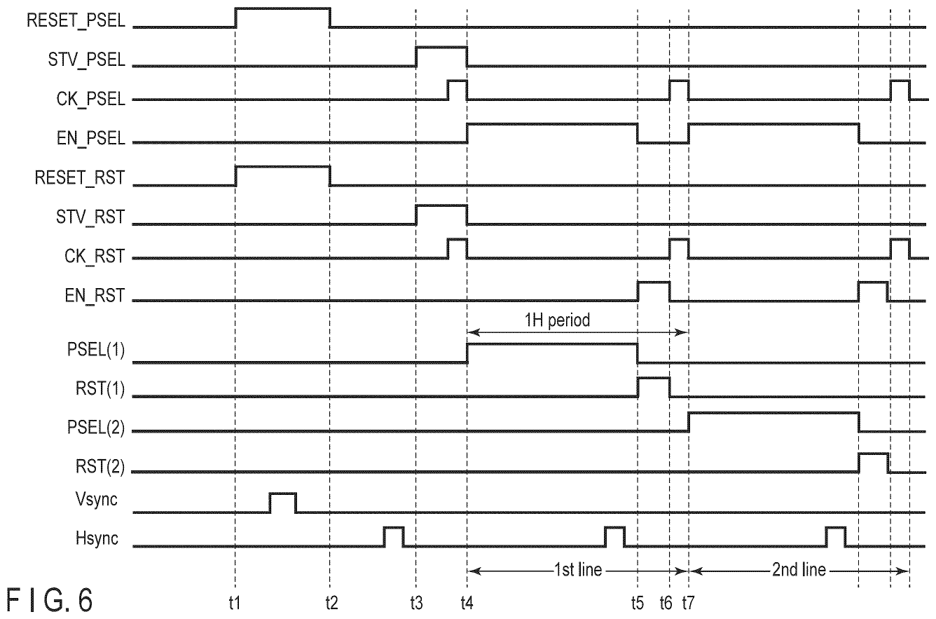

FIG. 6 is a signal waveform diagram to illustrate an example of an operation of the sensor device 2 according to the first embodiment.

The sensor control unit 10 outputs the frame reset signals RESET_PSEL and RESET_RST for a certain period of time (timing t1 to t2). The sensor control unit 10 outputs the vertical synchronization signal Vsync at a timing between timing t1 and t2.

The sensor control unit 10 supplies the frame reset signal RESET_PSEL to the PSEL gate driver 24 and supplies the frame reset signal RESET_RST to the RST gate driver 22. By the frame reset signals RESET_PSEL and RESET_RST, a potential Vn1 of the node n1 of the sensor unit P(i,j) is set (reset) to the reset voltage VCOM, and the output voltage Vo of the buffer amplifier 54 is set (reset) to a reset voltage (Vreset).

The sensor control unit 10 outputs the horizontal synchronization signal Hsync at a timing after timing t2.

The sensor control unit 10 outputs the frame start signals STV_PSEL and STV_RST for a certain period of time (timing t3 to t4). The sensor control unit 10 supplies the frame start signal STV_PSEL to the PSEL gate driver 24 and supplies the frame start signal STV_RST to the RST gate driver 22.

The sensor control unit 10 outputs the clock signals CK_PSEL and CK_RST after timing t3. The falling edge of the clock signals CK_PSEL and CK_RST is synchronized with the falling edge of the frame start signals STV_PSEL and STV_RST(timing t4). The sensor control unit 10 supplies the signal CK_PSEL to the PSEL gate driver 24 and the signal CK_RST to the RST gate driver 22. When the PSEL gate driver 24 and the RST gate driver 22 respectively receive the clock signals CK_PSEL and CK_RST, they select the sensor units P(1,1) to P(1,m) in the first row of the sensor units P arranged in a two-dimensional array.

The sensor control unit 10 supplies the enable signal EN_PSEL to the PSEL gate driver 24 for a certain period of time (timing t4 to t5). The PSEL gate driver 24 outputs the selection signal PSEL(1) in synchronization with the enable signal EN_PSEL. The selection signal PSEL(1) is supplied to the sensor units P(1, 1) to P(1,m) in the first row of the two-dimensional array. This turns on the MOSFET 36 of the sensor units P(1,1) to P(1,m) in the first row of the two-dimensional array. The drain-to-source current Ib of the MOSFET 34 flows to the current source 52. The voltage ΔVo of the node n1 is output from the sensor units P(1, 1) to P(1,m), and is supplied to the control units 10(1) to 10(m) as the voltage Vi, respectively. The photodiodes 40 of the sensor units P(1, 1) to P(1,m) in the first row are selected and the sensor signals Rx from the photodiodes are input to the sensor control unit 10 in a period of the selection signal PSEL(1) (timing t4 to t5).

The sensor control unit 10 outputs the horizontal synchronization signal Hsync at a timing prior to timing t5.

The sensor control unit 10 causes the A/D converter 56 to output a digital value corresponding to the sensor signal at timing t5. The sensor control unit 10 supplies the enable signal EN_RST to the RST gate driver 22 for a certain period of time (timing t5 to t6).

The RST gate driver 22 outputs the reset pulse RST(1) in synchronization with the enable signal EN_RST(timing t5 to t6). Timing t5 to t6 is the RST ON period. The reset pulse RST(1) is supplied to the sensor units P(1,1) to P(1,m) in the first row of the two-dimensional array. This turns on the MOSFET 32 of the sensor units P(1, 1) to P(1,m) in the first row of the two-dimensional array. The voltage at node n1 is reset to the voltage VCOM.

After timing t6, the sensor control unit 10 outputs the clock signals CK_PSEL and CK_RST to select a next row (the second row, the third row, . . . ) of the sensor units P(i,j) arranged in a two-dimensional array. The falling edge of the clock signals CK_PSEL and CK_RST is synchronized with the rising edge of the enable signal EN_PSEL (timing t7). The cycle of the clock signal CK_PSEL is equal to the cycle of the clock signal CK_RST and is referred to as a 1H period.

Timing t7 corresponds to timing t4. After timing t7, waveform changes similar to those of timings t4, t5, and t6 occur, and the sensor signals of the second row, are output.

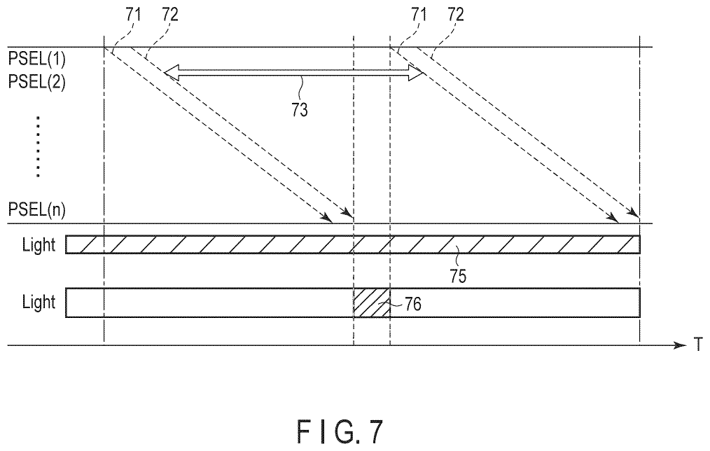

FIG. 7 illustrates an example of a light emission timing of the light source 5 according to the first embodiment. The sensor control unit 10 controls the light emission period of the light source 5 using the control signal. An exposure period of the sensor panel 4 may be a whole period as illustrated by “75” or a period from the rising edge 72 of the selection signal PSEL(n) in the nth row to the rising edge 71 of the reset signal RST(1) in the first row as illustrated by “76”.

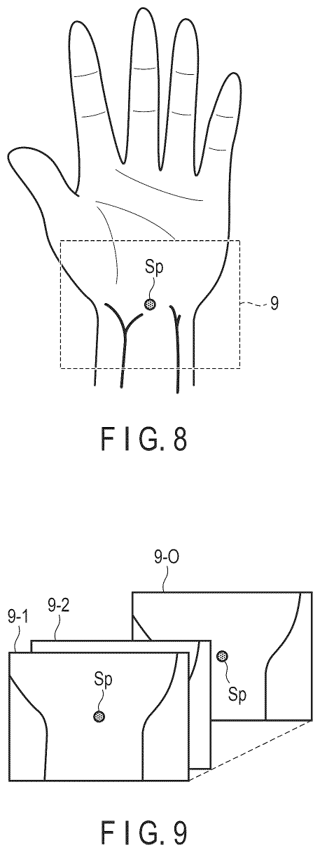

FIG. 8 to FIG. 12 illustrate an example of the sensing principle of the sensor device 2 according to the first embodiment.

FIG. 8 shows an example of mounting the sensor device 2 including the sensor panel 4 and the light source 5 to the artery of the palm (or back of the hand).

FIG. 9 shows an example of acquiring the sensor signal Rx in which the signal processor 11 acquires the sensor signal Rx at position Sp for multiple frames 9-1, 9-2, . . . 9-o, where o is any positive integer. The position Sp is determined based on the synchronization signal Vsync and the horizontal synchronization signal Hsync. The position Sp may be freely determined. The position Sp may be the center of the frame, or may be a point where biometric information is easy to detect, which is determined by trial and error.

The sensor signal Rx (output voltage Vo of the buffer amplifier 54) of multiple frames changes depending on a type of the sensor device 2, the presence, or absence of the sensing target, and the state of the sensing target.

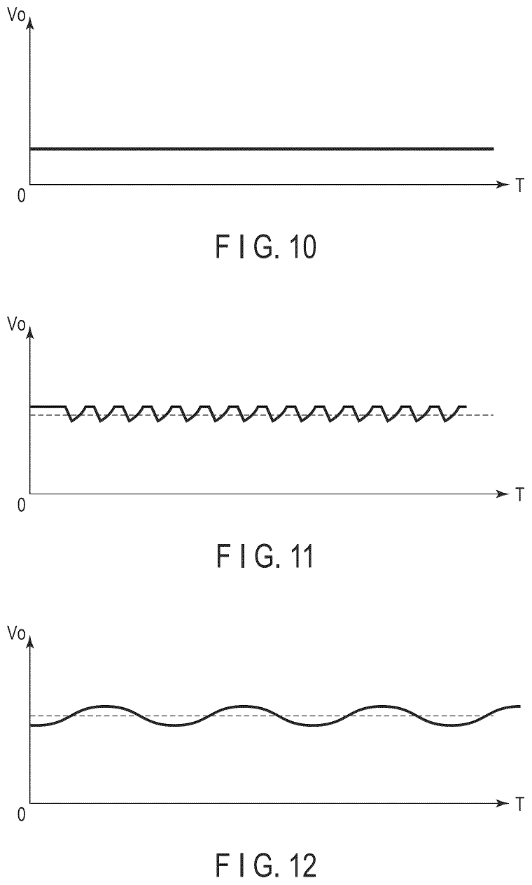

In a case where the sensor device 2 is a transmissive sensor and is not attached to the sensing target, i.e., in a case where the sensor device 2 does not detect the sensing target, the luminance of the light incident on the sensor panel 4 is high. Therefore, the photocurrent Id of the photodiode 40 is large and the output voltage Vo of the buffer amplifier 54 is a low constant voltage (FIG. 10).

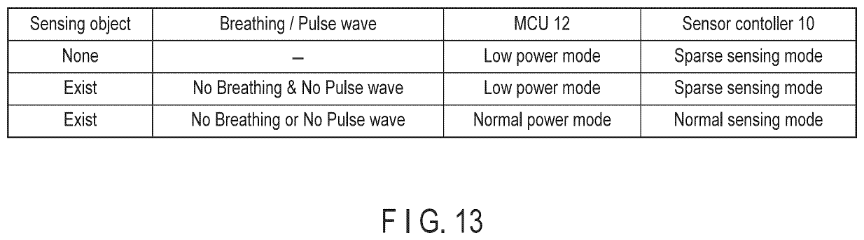

In a case where the sensor device 2 is a reflective sensor and is not attached to the sensing target, the luminance of the light incident on the sensor panel 4 is low. Therefore, the photocurrent Id of the photodiode 40 is small and the output voltage Vo of the buffer amplifier 54 is a high constant voltage (dashed line in FIGS. 11 and 12).

In the case where the sensor device 2 is attached to the sensing target, the output voltage Vo of the buffer amplifier 54 fluctuates according to the state of the sensing target (pulse detection, breathing detection). In a case where the sensor device 2 detects the pulse of the sensing target, the output voltage Vo of the buffer amplifier 54 fluctuates around the high constant voltage in a short cycle (cycle of approximately 50 to 120 bpm) (solid line in FIG. 11). In a case where the sensor device 2 detects the breathing of the sensing target, the output voltage Vo of the buffer amplifier 54 fluctuates around the high constant voltage in a long cycle (cycle of approximately 12 to 50 bpm) (solid line in FIG. 12).

FIG. 13 is a diagram illustrating an example of an overview of the operation of the sensor device 2 according to the first embodiment. The MCU 12 can operate in a normal power mode or a low power mode. The selection of the normal power mode or the low power mode is based on the status data and biometric data from the signal processor 11. The MCU 12 operating in the normal power mode is capable of performing all functions. The MCU 12 operating in the low power mode is not capable of performing some functions. For example, the MCU 12 operating in the normal power mode can read sensor data supplied by sensor control unit 10 and transfer it to the PC 3 via the USB IF circuit 13. However, the MCU 12 operating in the low power mode cannot perform this reading and transfer. The power consumption of the MCU 12 is less in the low power mode than in the normal power mode.

The sensor panel 4 can operate in a normal sensing mode or a sparse sensing mode. The selection of the sensing mode is based on the control signal from the sensor control unit 10. The sensor panel 4 operating in the normal sensing mode senses continuously over multiple frame periods. The sensor panel 4 operating in the sparse sensing mode senses for one or a few multiple frame periods. The sensing of one or a few multiple frame periods is repeated intermittently. The power consumption of the sensor control unit 10 and the sensor panel 4 is less in the sparse sensing mode than in the normal sensing mode.

In a case where there is no sensing target present, the MCU 12 operates in the low power mode, regardless of the presence or absence of breathing/pulse, and the sensor control unit 10 sets the sensing mode of the sensor panel 4 to the sparse sensing mode.

In a case where the sensing target is present, but neither breathing nor pulse is detected, the MCU 12 operates in the low power mode, and the sensor control unit 10 sets the sensing mode of the sensor panel 4 to the sparse sensing mode.

In a case where the sensing target is present and breathing or pulse is detected, the MCU 12 operates in the normal power mode, and the sensor control unit 10 sets the sensing mode of the sensor panel 4 to the normal sensing mode.

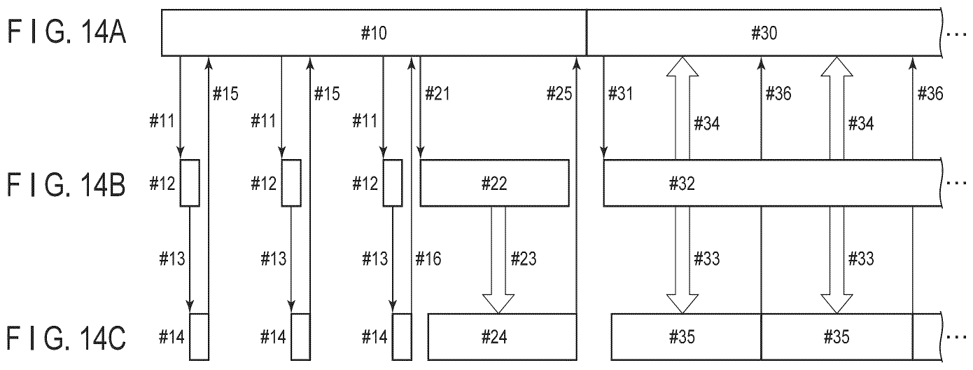

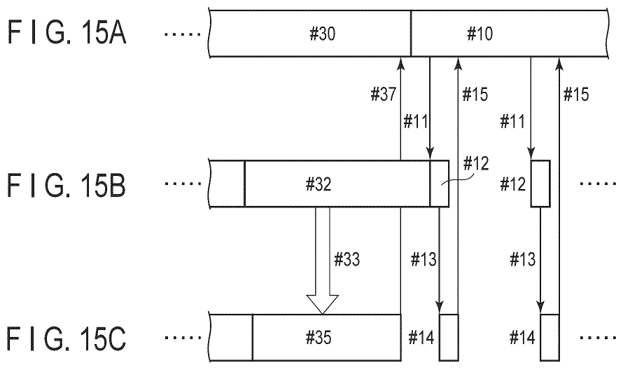

FIG. 14A, FIG. 14B, and FIG. 14C illustrate an example of details of the operation of the sensor device 2 according to the first embodiment. FIG. 15A, FIG. 15B, and FIG. 15C illustrate an example of details of the operation of the sensor device 2 according to the first embodiment following FIG. 14A, FIG. 14B, and FIG. 14C. FIG. 14A and FIG. 15A describe the operating mode of the MCU 12. FIG. 14B and FIG. 15B describe the operation of the sensor control unit 10 (sensing mode of the sensor panel 4). FIG. 14C and FIG. 15C describe the processing of the signal processor 11.

The MCU 12 operates in the low power mode (#10) when the power of the sensor device 2 is turned on. During the low power mode operation, the MCU 12 transmits the sparse sensing mode signal to the sensor control unit 10 (#11).

When the sensor control unit 10 receives the sparse sensing mode signal (#11), it sets the sensing mode of the sensor panel 4 to the sparse sensing mode and causes the sensor panel 4 to perform sparse sensing for one frame (#12). Note that the number of frames for one sparse sensing is not limited to one frame, but may be two or three frames.

The sensor control unit 10 transmits the sensor signal voltage Vo of the position Sp output from the sensor unit P(i,j) by the sparse sensing (#12) of the sensor panel 4 to the signal processor 11 (#13).

The signal processor 11 determines the presence or absence of a sensing target based on the voltage Vo (#14). At the timing of #14, the sensor device 2 is not attached to the sensing target. Therefore, the signal processor 11 transmits a determination result of no sensing target to the MCU 12 (#15).

When the MCU 12 receives the determination result of no sensing target (#15), it maintains the low power mode (#10) and transmits the sparse sensing mode signal to the sensor control unit 10 (#11). Hereinafter, processes #12 to #15 are repeated. FIGS. 14A, 14B, and 14C show examples where no sensing target was detected in the second sparse sensing, and the sensing target was detected in the third sparse sensing.

After the third sparse sensing, the signal processor 11 transmits the determination result indicating that there is a sensing target to the MCU 12 (#16).

When the MCU 12 receives the determination result indicating that there is a sensing target (#16), it maintains the low power mode (#10) and transmits a normal sensing mode signal to the sensor control unit 10 (#21). Note that, for convenience of illustration, there is a time difference between the reception of the determination result (#16) and the transmission of the normal sensing mode signal (#21), but the normal sensing mode signal may be transmitted immediately after the reception of the determination result.

When the sensor control unit 10 receives the normal sensing mode signal (#21), it sets the sensing mode of the sensor panel 4 to the normal sensing mode and causes the sensor panel 4 to perform a continuous sensing for a certain period of time (e.g., 10 seconds) (#22). The signal processor 11 needs a sense signal of a predetermined number of frames to detect the state of the sensing target and biometric information. The certain period of time is a period required for sensing the predetermined number of frames.

The sensor control unit 10 transmits the sensor signal voltage Vo of the position Sp of the predetermined number of frames output from the sensor unit P(i,j) by the normal sensing (#22) of the sensor panel 4 to the signal processor 11 (#23).

The signal processor 11 detects the presence or absence of a pulse wave, the presence or absence of breathing, and the pulse rate or breathing rate based on the time variation of the sensor signal voltage Vo of the predetermined number of frames (#24). The signal processor 11 detects the presence or absence of the pulse wave or breathing, but may not detect the pulse rate or breathing rate. When the signal processor 11 detects the presence or absence of the pulse wave or breathing, or detects the pulse rate or breathing rate, a detection signal is transmitted to the MCU 12 (#25).

When the MCU 12 receives the detection signal (#25), it changes the operating mode to the normal power mode (#30). During the normal power mode operation (#30), the MCU 12 transmits the normal sensing mode signal to the sensor control unit 10 (#31). Note that, for convenience of illustration, there is a time difference between the reception of the detection signal (#25) and the transmission of the normal sensing mode signal (#31), but the normal sensing mode signal may be transmitted immediately after reception of the detection signal.

If the MCU 12 does not receive the detection signal within a certain period of time after transmitting the normal sensing mode signal (#21), it maintains the low power mode and transmits the sparse sensing mode signal to the sensor control unit 10.

When the operating mode is changed to the normal power mode (#30), the MCU 12 transmits the normal sensing mode signal to the sensor control unit 10 (#31).

When the sensor control unit 10 receives the normal sensing mode signal (#31), it sets the sensing mode of the sensor panel 4 to the normal sensing mode and causes the sensor panel 4 to perform a continuous sensing (#32). The sensor control unit 10 transmits the sensor signal voltage Vo of the position Sp of each frame output from the sensor unit P(i,j) by the normal sensing of the sensor panel 4 to the signal processor 11 (#33), and transmits the sensor signal voltage Vo of the position Sp of each frame to the MCU 12 (#34).

Each time the signal processor 11 receives the sensor signal voltage Vo of the predetermined number of frames, it detects the presence or absence of the pulse wave, the presence or absence of breathing, the pulse rate, or breathing rate based on the time variation of the sensor signal voltage Vo (#35). When the signal processor 11 detects the pulse rate or breathing rate, it transmits the detected number data to the MCU 12 (#36).

When the MCU 12 receives the detected number data (#36), it maintains the normal power mode (#30) and transmits the sensor signal voltage Vo of the position Sp of each frame and the detected number data each time sensing is performed of the predetermined number of frames to the PC 3 via the USB IF circuit 13 (see FIG. 14A, FIG. 14B, and FIG. 14C).

As shown in FIG. 15A, FIG. 15B, and FIG. 15C, in a case where the pulse rate or breathing rate cannot be detected by the processing (#35) based on the sensor signal voltage Vo of the predetermined number of frames, the signal processor 11 transmits a detection failure signal to the MCU 12 (#37).

When the MCU 12 receives the detection failure signal (#37), it changes the operating mode from the normal power mode to the low power mode (#10) and transmits the sparse sensing mode signal to the sensor control unit 10 (#11). Hereinafter, processes #11 to #15 or processes #11 to #16 are executed in the same manner as in the initial stage of FIG. 14A, FIG. 14B, and FIG. 14C.

The sensor device 2 according to the first embodiment detects the presence or absence of the sensing target and the state of the sensing target. In the case where no sensing target is detected, the MCU 12 operates in the low power mode and the sensor control unit 10 causes the sensor panel 4 to perform the sparse sensing.

When the sensing target is detected, the sensor control unit 10 sets the sensing mode of the sensor panel 4 to the normal sensing mode and causes the sensor panel 4 to perform normal sensing for a certain period of time. The signal processor 11 determines the presence or absence of the pulse wave or breathing based on the changes in the sense signal voltage of multiple frames obtained as a result of the normal sensing for a certain period of time. When the pulse wave or breathing is detected, the MCU 12 starts an operation in the normal power mode, and the sensor control unit 10 causes the sensor panel 4 to perform a continuous sensing. The signal processor 11 obtains biometric information such as the breathing rate and the pulse rate for each sensing of a predetermined number of frames during the continuous sensing. The sensor signal voltage obtained by the continuous sensing is transmitted to the PC 3.

In this way, the sparse sensing is performed until the sensing target is detected, and once the sensing target is detected, the normal sensing is performed. When the breathing or pulse is detected, the operating mode of the MCU 12 is changed to the normal power mode and the MCU 12 is activated. Thus, power consumption can be reduced to the necessary minimum.

### Second Embodiment

The overall configuration of a second embodiment is the same as the first embodiment shown in FIG. 1. FIG. 1 to FIG. 7, FIG. 13, FIG. 14A to FIG. 14C, and FIG. 15A to FIG. 15C also apply to the second embodiment.

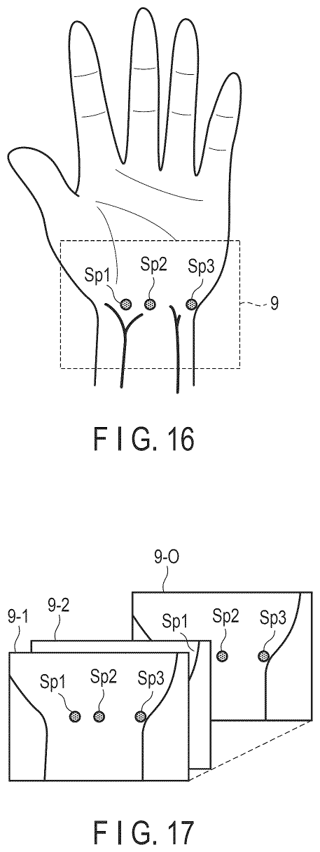

FIG. 16 illustrates an example of mounting the substrate 9 including the sensor panel 4 and the light source 5 to the sensing target according to the second embodiment. FIG. 16 corresponds to FIG. 8. The substrate 9 is mounted on the artery of the palm (or the back of the hand).

The signal processor 11 acquires sensor signals Rx1, Rx2, and Rx3 for each of the multiple positions (e.g., three positions) Sp1, Sp2, and Sp3 for multiple frames 9-1, 9-2, 9-o (FIG. 17). The positions Sp1, Sp2, and Sp3 are determined based on the synchronization signal Vsync and the horizontal synchronization signal Hsync. The positions Sp1, Sp2, and Sp3 may be freely determined. The positions Sp1, Sp2, and Sp3 may be included in the central region of the frame, or they may be points where biometric information is easy to detect, which are obtained by trial and error.

As in the first embodiment, the sensor signals Rx1, Rx2, and Rx3 (output voltages Vo1, Vo2, and Vo3 of the buffer amplifier 54) of the multiple frames change depending on the presence or absence of a sensing target and the state of the sensing target. In a case where the sensor device 2 is not attached to the sensing target, the output voltages Vo1, Vo2, and Vo3 of the buffer amplifier 54 are low constant voltages as in FIG. 10.

In a case where the sensor device 2 is attached to the sensing target, the output voltages Vo1, Vo2, and Vo3 of the buffer amplifier 54 are high constant voltages as shown in the dashed lines of FIG. 11.

In a case where the sensor device 2 detects the pulse of the sensing target, the output voltage Vo of the buffer amplifier 54 fluctuates around the high constant voltage in a short cycle (cycle of approximately 50 to 120 bpm) (solid line in FIG. 11). In a case where the sensor device 2 detects the breathing of the sensing target, the output voltage Vo of the buffer amplifier 54 fluctuates around the high constant voltage in a long period (cycle of approximately 12 to 50 bpm) (solid line in FIG. 12).

One example of the details of the operation of the sensor device 2 according to the second embodiment is similar to the operation of the sensor device according to the first embodiment shown in FIG. 14A to FIG. 14C and FIG. 15A to 15C. The operation of the sensor device according to the second embodiment is described with reference to FIG. 14A to FIG. 14C and FIG. 15A to FIG. 15C.

The MCU 12 operates in the low power mode (#10) when the power of the sensor device 2 is turned on. During the low power mode operation (#10), the MCU 12 transmits the sparse sensing mode signal to the sensor control unit 10 (#11).

When the sensor control unit 10 receives the sparse sensing mode signal (#11), it sets the sensing mode of the sensor panel 4 to the sparse sensing mode and causes the sensor panel 4 to perform the sparse sensing for one frame (#12).

The sensor control unit 10 transmits the sensor signal voltages Vo1, Vo2, and Vo3 of each of the positions Sp1, Sp2, and Sp3 output from the sensor unit P(i,j) by the sparse sensing (#12) of the sensor panel 4 to the signal processor 11 (#13).

The signal processor 11 determines the presence or absence of the sensing target based on the voltages Vo1, Vo2, and Vo3 (#14). In a case where any one of the three determination results based on the voltages Vo1, Vo2, and Vo3 indicates that there is a sensing target, the signal processor 11 finally determines that there is a sensing target.

The signal processor 11 transmits a determination result of no sensing target (#15), or transmits a determination result indicating that there is a sensing target (#16) to the MCU 12.

When the MCU 12 receives the determination result of no sensing target (#15), it maintains the low power mode and transmits the sparse sensing mode signal to the sensor control unit 10 (#11). Hereinafter, processing #12 to #15 are repeated. After the third sparse sensing, the signal processor 11 transmits a determination result indicating that there is a sensing target to the MCU 12 (#16).

When the MCU 12 receives the determination result indicating that there is a sensing target (#16), it maintains the low power mode (#10) and transmits the normal sensing mode signal to the sensor control unit 10 (#21).

When the sensor control unit 10 receives the normal sensing mode signal (#21), it sets the sensing mode of the sensor panel 4 to the normal sensing mode, and causes the sensor panel 4 to perform a continuous sensing for a certain period of time (e.g., 10 seconds) (#22).

The sensor control unit 10 transmits the sensor signal voltages Vo1, Vo2, and Vo3 of the three positions Sp1, Sp2, and Sp3 of a predetermined number of frames output from the sensor unit P(i,j) by the normal sensing of the sensor panel 4 to the signal processor 11 (#23).

The signal processor 11 detects the presence or absence of the pulse wave, the presence or absence of breathing, and the pulse rate or breathing rate based on the time variation of the sensor signal voltages Vo1, Vo2, and Vo3 for the predetermined number of frames (#24). When the signal processor 11 detects the presence or absence of the pulse wave or breathing, or the pulse rate or breathing rate based on one of the sensor signal voltages Vo1, Vo2, and Vo3, it transmits a detection signal to the MCU 12 (#25). Since the sensor panel 4 detects the presence or absence of the pulse wave, the presence or absence of the breathing, and the pulse rate or breathing rate based on one of the multiple positions Sp1, Sp2, or Sp3, even if the sensor signal voltage cannot be detected correctly at any two of the positions Sp1, Sp2, or Sp3, the sensor panel 4 can correctly detect the sensor signal voltage at the remaining one position.

When the MCU 12 receives the detection signal (#25), it changes the operating mode to the normal power mode (#30). During the normal power mode operation (#30), the MCU 12 transmits the normal sensing mode signal to the sensor control unit 10 (#31).

If the MCU 12 does not receive the detection signal within a certain period of time after transmitting the normal sensing mode signal (#21), it maintains the low power mode and transmits the sparse sensing mode signal to the sensor control unit 10.

When the operating mode is changed to the normal power mode (#30), the MCU 12 transmits the normal sensing mode signal to the sensor control unit 10 (#31).

When the sensor control unit 10 receives the normal sensing mode signal (#31), it sets the sensing mode of the sensor panel 4 to the normal sensing mode and causes the sensor panel 4 to perform a continuous sensing (#32). The sensor control unit 10 transmits the sensor signal voltages Vo1, Vo2, and Vo3 of the positions Sp1, Sp2, and Sp3 of each frame output from the sensor unit P(i,j) by the normal sensing of the sensor panel 4 to the signal processor 11 (#33), and transmits the sensor signal voltages Vo1, Vo2, and Vo3 of the positions Sp1, Sp2, and Sp3 of each frame to the MCU 12 (#34).

Each time the signal processor 11 receives the sensor signal voltages Vo1, Vo2, and Vo3 of the predetermined number of frames, it detects the presence or absence of the pulse wave, the presence or absence of breathing, and the pulse rate or breathing rate based on the time variation of the sensor signal voltages Vo1, Vo2, and Vo3 (#35). When the signal processor 11 detects the presence or absence of the pulse wave or breathing based on one of the sensor signal voltages Vo1, Vo2, and Vo3, it transmits the detected number data to the MCU 12 (#36). The signal processor 11 may determine the presence or absence of the pulse wave, the presence or absence of breathing, and the pulse rate or breathing rate in process #35 based on the voltages Vo1, Vo2, and Vo3 used for the determination in process #25.

When the MCU 12 receives the detected number data (#36), it maintains the normal power mode (#30) and transmits the sensor signal voltage Vo of the position Sp of each frame and the detected number data each time sensing is performed of the predetermined number of frames to the PC 3 via the USB IF circuit 13 (see FIG. 14A to FIG. 14C).

As shown in FIG. 15A to FIG. 15C, in a case where the pulse rate or breathing rate cannot be detected by the processing (#35) based on the sensor signal voltages Vo1, Vo2, and Vo3 of the predetermined number of frames, the signal processor 11 transmits a detection failure signal to the MCU 12 (#37).

When the MCU 12 receives the detection failure signal (#37), it changes the operating mode from the normal power mode (#30) to the low power mode (#10) and transmits the sparse sensing mode signal to the sensor control unit 10 (#11). Hereinafter, processes #12 to #15 or processes #12 to #16 are executed in the same manner as in the initial stage of FIG. 14A to FIG. 14C.

The sensor device 2 according to the second embodiment detects the sense signal voltage at multiple points in one frame, and detects the presence or absence of the sensing target and the state of the sensing target based on any of the multiple sense signal voltages. Therefore, detection accuracy can be increased.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

What is claimed is:

1. A sensor device comprising:
a sensor configured to detect reflected light from a sensing target or transmitted light of the sensing target;
a control unit configured to cause the sensor to perform sensing and receive a detection signal from the sensor; and
a processor configured to determine presence or absence of the sensing target based on a detection signal of the sensor and obtain biometric information of the sensing target based on fluctuations in the detection signal over a certain period of time, wherein
the control unit is configured to cause the sensor to perform first sensing;
the control unit is configured to cause the sensor to perform second sensing when the processor determines the presence of the sensing target during the first sensing; and
power consumption during the first sensing is less than power consumption during the second sensing.

2. The sensor device of claim 1, further comprising:
a transmission unit configured to receive the detection signal and transmits the detection signal to an external device, wherein
the transmission unit is configured to operate in low power mode; and
the transmission unit is configured to operate in normal power mode when the processor detects a fluctuation in the detection signal.

3. The sensor device of claim 1, wherein
the control unit is configured to cause the sensor to periodically perform the first sensing until the processor determines the presence of the sensing target;
the first sensing is performed by a first period; and
the second sensing is performed by a second period, which is longer than the first period.

4. The sensor device of claim 3, wherein
the control unit is configured to cause the sensor to continuously perform the second sensing until the processor determines that there is no sensing target.

5. The sensor device of claim 1, wherein
the biometric information is indicative of a breathing rate or a pulse rate.

6. The sensor device of claim 1, wherein
the sensor comprises a plurality of units arranged in a one-dimensional or two-dimensional array;
each of the plurality of units comprises:
a photodiode configured to generates a current in response to the reflected light or the transmitted light wherein an end of the photodiode is connected to an output terminal of the unit;
a capacitor charged by the current;
a first transistor configured to set a voltage at the end of the photodiode to a reset voltage;
a second transistor configured to flow a constant current in accordance with a charged voltage of the capacitor; and
a third transistor configured to be conductive for a certain period of time and output the constant current from the unit.

7. The sensor device of claim 6, wherein
the first transistor includes a first metal oxide semiconductor type field effect transistor (MOSFET) connected between a terminal of the reset voltage and the end of the photodiode;
the capacitor is connected in parallel to the photodiode;
the second transistor includes a second MOSFET;
a gate of the second MOSFET is connected to the end of the photodiode; and
the third transistor includes a third MOSFET connected between the second MOSFET and the output terminal.

8. A sensing method for a sensor device comprising:
a sensor configured to detect reflected light from a sensing target or transmitted light of the sensing target;
a control unit configured to cause the sensor to perform sensing and receive a detection signal from the sensor; and
a processor configured to determine presence or absence of the sensing target based on a detection signal of the sensor and obtain biometric information of the sensing target based on fluctuations in the detection signal over a certain period of time,
the method comprising:
causing by the control unit the sensor to perform first sensing; and
causing by the control unit the sensor to perform second sensing when the processor determines the presence of the sensing target during the first sensing, wherein power consumption during the first sensing is less than power consumption during the second sensing.
